(19) United States
(12) Patent Application Publication
(10) Pub. No.: US 2015/0351726 A1
(43) Pub. Date: Dec. 10, 2015

(54) User event-based optimization of B-mode ultrasound imaging

(71) Applicant: Siemens Medical Solutions USA, Inc., Malvern, PA (US)

(72) Inventors: Manoj G. Menon, Bellevue, WA (US); King Yuen Wong, Issaquah, WA (US)

(21) Appl. No.: 14/297,355

(22) Filed: Jun. 5, 2014

(51) Int. Cl. A61B 8/08

(52) U.S. Cl. CPC A61B 8/5292

(57) ABSTRACT

B-mode ultrasound imaging is optimized for a user's region of interest. For B-mode imaging, a region of interest is determined. Some indication of a location or locations of interest to the user in the B-mode image is used to identify the region of interest. The parameters used for B-mode imaging are then set based on the identified region of interest rather than an entire B-mode field of view. Subsequent B-mode imaging is performed with these parameters optimized for the region.

### BACKGROUND

The present invention relates to B-mode ultrasound imaging. In particular, optimization of B-mode ultrasound imaging is provided.

A number of B-mode imaging parameters may improve image quality. The large number of controls available to the user may slow exam times and introduce complication. Therefore, it is useful to automatically set the parameters based on image data. Gain, frequency, persistence, smoothing, power, gray levels, and dynamic range are examples of automatically set parameters. However, the automated setting of parameters may provide sub-optimal image quality for some tissues of interest within the image.

Ultrasound imaging typically includes B-mode, color flow mode, and spectral Doppler imaging. In color flow mode, the user places a box as a region of interest (ROI) for color flow in the B-mode image. The system uses a set of parameters chosen based on the position and size of the region of interest for color flow imaging in the entire region of interest. Similarly, for spectral Doppler imaging, a gate is chosen by the user. The gate size and position are used to automatically set imaging parameters. In the case of B-mode ultrasound images, a region of interest for the B-mode itself is not chosen as the regions of interest are for the gate or color flow.

### BRIEF SUMMARY

By way of introduction, the preferred embodiments described below include methods and systems for optimization in B-mode ultrasound imaging. For B-mode imaging, a region of interest is determined. Some indication of a location or locations of interest to the user in the B-mode image is used to identify the region of interest. The parameters used for B-mode imaging are then set based on the identified region of interest rather than an entire B-mode field of view. Subsequent B-mode imaging is performed with these parameters optimized for the region.

In a first aspect, a method of optimization in B-mode ultrasound imaging is provided. An ultrasound system generates a first B-mode image of a scan region of a patient. The ultrasound system receives an indication of a user's location of interest in the B-mode image. A processor identifies the region of interest from the indication of the user's location of interest. The region of interest is a sub-set of a B-mode scan region. At least one B-mode imaging parameter is set as a function of the region of interest. The ultrasound system, using the setting of the at least one B-mode imaging parameter, generates a second B-mode image of the scan region of the patient. The second B-mode image is free of a graphic showing the region of interest.

In a second aspect, a system is provided for optimization in B-mode ultrasound imaging. An ultrasound imager includes a B-mode detector configured to detect B-mode data. The ultrasound imager is configured to generate a first B-mode image as a function of a value of a variable. A user input is configured to receive an input of a region of the first B-mode image. The region is a focus of the user. A processor is configured to change the value of the variable based on the region and cause the ultrasound imager to generate a second B-mode image as a function of the changed value of the variable.

In a third aspect, a non-transitory computer readable storage medium has stored therein data representing instructions executable by a programmed processor for optimization in B-mode ultrasound imaging. The storage medium includes instructions for receiving a user input of a change in focal position, field of view depth, an imaging frequency, or combinations thereof; determining a region of interest that is a sub-set of a field of view for B-mode scanning, the determining of the region of interest being a function of the user input; changing a B-mode parameter as a function of the region of interest; and generating a B-mode image using the changed B-mode parameter, the B-mode parameter used for the entire B-mode image.

The present invention is defined by the following claims, and nothing in this section should be taken as limitations on those claims. Further aspects and advantages of the invention are disclosed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for optimization in B-mode ultrasound imaging;

FIG. 2 illustrates example inputs and a region of interest relative to a B-mode image; and

FIG. 3 is a block diagram of one embodiment of a system for optimization in B-mode ultrasound imaging.

### DETAILED DESCRIPTION OF THE DRAWINGS AND PRESENTLY PREFERRED EMBODIMENTS

A region of interest in B-mode imaging is predicted based on a user event. The prediction is based on user indication. Example indications include change in frequency, change in focal position, change in depth of field of view, retinal tracking, and/or user input of the region itself. Where multiple indications of the B-mode region of interest are received, the relevance and/or timing of the indications are used to determine the B-mode region of interest. The indication is used to find the subset of the image that should be used for automatic or predetermined optimization.

The B-mode region of interest is used for optimization of the B-mode imaging for the entire B-mode field of view, not just the region of interest. The region of interest within the currently displayed image is determined for analysis and/or special consideration to choose more optimal imaging parameters for subsequent image frames. For example, if the user is performing a renal exam for kidney imaging, the user may have to increase the imaging depth or lower the frequency to improve penetration to visualize the entire kidney. Although the depth and frequency are modified for the entire image, it can be assumed that the user region of interest for B-mode imaging is located in the lower part of the image. One or more B-mode imaging parameters are set based on the region of interest being in the lower part of the image.

User event weighted automatic optimization in B-mode imaging may improve plunkability. By automating B-mode optimization based on a user indication, more consistent or appropriate image quality may be provided.

FIG. 1 shows one embodiment of a method for optimization in B-mode ultrasound imaging. In general, a user's region of interest in a B-mode image is determined. The region of interest is then used to set one or more B-mode imaging parameters so that subsequent B-mode images are optimized to the region of interest.

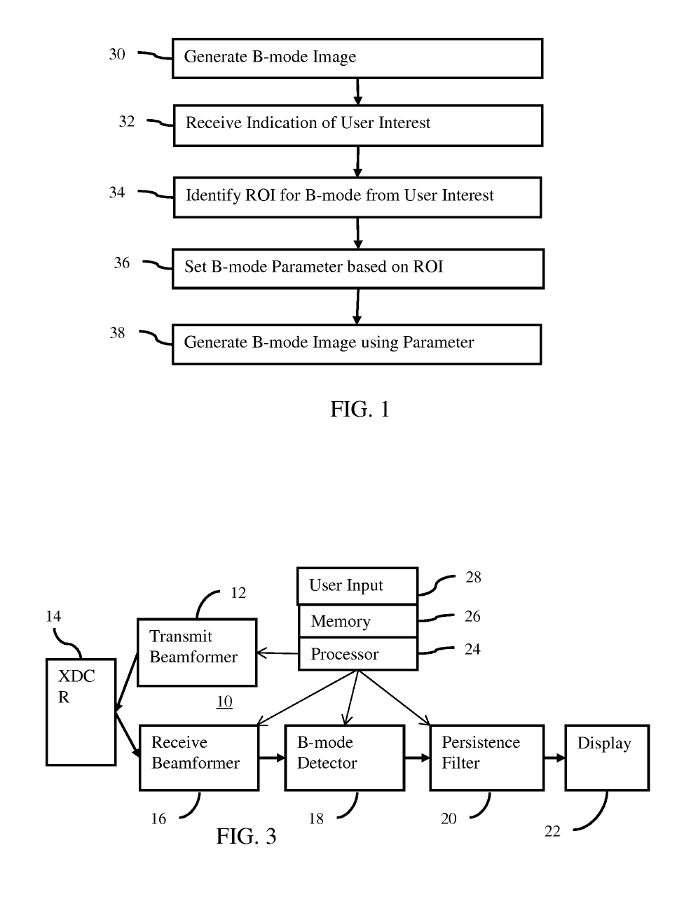

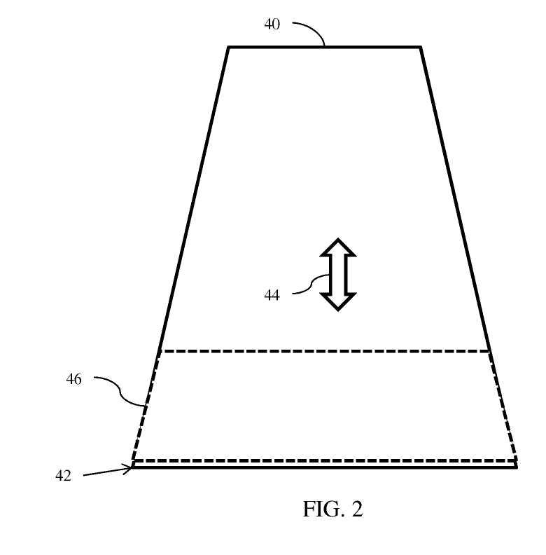

The method is performed by the system shown in FIG. 3 or a different system. For example, a medical diagnostic ultrasound imaging system generates B-mode images. The imaging system receives indications of the user's region of interest from a user input or attached sensors. A processor of the imaging system determines the region of interest, automatically sets one or more parameters based on the region of interest, and/or causes the imaging system to perform B-mode imaging with the one or more parameters as set based on the region of interest. Imaging systems, processors, and/or user input devices are used to perform any one or more of the acts.

The acts are performed in the order shown or another order. Additional, different or fewer acts may be used. For example, act 34 is subsumed into act 32 where the indication is of the region of interest rather than a spatial location used to derive the region of interest. As another example, acts for weighting or combining multiple indications during a given imaging session or imaging configuration (e.g., scanning of a given patient) and using the weighted or combined indications to find the region of interest are provided.

In act 30, a B-mode image is generated. To scan a field of view with ultrasound, transmit and receive beams are formed by an ultrasound system for B-mode imaging. FIG. 2 shows an example field of view 40. The responsive data represents samples in the field of view. The scanning is in any format, such as sector, linear, or Vector®.

The transmit and/or receive beam characteristics may be set or responsive to parameters. The depth and/or lateral extent of the field of view for the B-mode imaging is set. Similarly, the transmit beam focal depth, transmit frequency, receive frequency, line density, transmit waveform (e.g., number of cycles and/or envelope shape), frame rate, and/or other scanning characteristics are set. The number of transmit focal positions per scan line (e.g., one or two) may be set. Different, additional, or fewer scan (e.g., transmit and receive) parameters may be used.

Data received from the scanning is detected. A B-mode detector determines the intensity of acoustic echoes represented by the received data. For example, the receive data is formatted as in-phase and quadrature data. A square root of a sum of the squares of the in-phase and quadrature terms is calculated as the intensity. Other measures of the magnitude of the acoustic echo may be used for B-mode detection.

Other B-mode processing may be performed based on values for parameters. For example, the detected B-mode data is spatially filtered. One or more parameters may establish the amount, type, or other filtering characteristic. As another example, a sequence of frames from a corresponding sequence of scans of the entire field of view is acquired. Different pairs or other sized groupings of the resulting B-mode frames of data are temporally filtered. Infinite impulse or finite impulse response filtering may be used. One or more parameters may establish the amount, type, or other filtering characteristic. In another example, a general or overall gain is applied. One or more parameters may establish the overall gain. Additionally or alternatively, depth dependent gains may be applied. Different, additional, or fewer B-mode processing parameters may be used.

The values of the parameters for B-mode scanning are initially set using any process. In one embodiment, one or more of the parameters are set based on input by the user, predetermined values, and/or selection of an application or configuration. In alternative or additional embodiments, one or more of the parameters are set based on feedback or adapting to the data received from the scanning. Automatic setting of the value or values of the parameter or parameters is performed. For example, the overall gain and/or dynamic range of the B-mode data is set based on identifying B-mode data for locations associated with tissue in the field of view and using an average, median or other B-mode intensity for the tissue locations to set the gain and/or dynamic range.

After processing, the detected B-mode data is scan converted, if needed, and displayed. A B-mode image is generated. The B-mode image represents the intensity or strength of return of acoustic echoes in the B-mode field of view. The intensities or B-mode data is mapped to gray scale within the dynamic range of the display. The gray scale may be equal or similar red, green, blue (RGB) values used by the display to control pixels. Any color or gray scale mapping may be used. One or more parameters may control the mapping.

The B-mode image includes information of interest to the user and may include information not of interest. For example, in B-mode imaging of the liver, the shallow depths may include other organs and/or skin. This information is not of interest, but the deeper representation or B-mode information for the liver is of interest to the user. Conversely, more shallow locations may be of more interest. Rather than or in addition to axial (e.g., depth) spacing, the information of interest may be at different lateral locations.

Instead of B-mode imaging configured for the field of view, which includes information of particular interest and information of lesser interest, the B-mode imaging may be configured for the user's region of interest. The scanning and/or B-mode processing parameters for the entire B-mode image may be optimized to provide desired quality at the region of interest rather than being based on the entire field of view.

In act 32, an indication of a user's location of interest in the B-mode image is received. For subsequent B-mode imaging during a same session, for a same patient, and/or later times in real-time imaging, the ultrasound system receives an indication of one or more locations associated with user interest as compared to locations associated with less or no user interest. The location or locations may be a point, line, or area of the field of view.

Typical B-mode imaging does not have a region of interest designated by a graphic box or other shape by the user. Where a region of interest graphic is provided on the B-mode image, the region of interest is to designate the region for color Doppler or spectral Doppler imaging. The region is of interest for other modes, not the B-mode. The information from the other modes may entirely or partially obscure, replace, or cover the B-mode information.

A separate indication than a color pan box (i.e., Doppler region of interest or gate location) may be provided for the B-mode imaging. Any indication of the location or locations of user interest for B-mode imaging may be used. For example, the user changes an imaging depth, number of focal positions per transmit line, or the field of view depth. By increasing the depth, the user indicates that deeper locations in the field of view are important or more important. Similarly, altering a transmit frequency to be lower results in less resolution but greater depth of penetration, indicating deeper locations as important. The alteration of the transmit focus 44 to be deeper or adding a set of deeper foci likewise indicates importance of deeper locations. Conversely, higher transmit frequency, use of harmonic receive frequencies, setting the field of view depth 42 to be more shallow, and/or setting the transmit focus 44 to be shallower may indicate that shallower locations are important to the user. Changing the number of focal positions may indicate that deeper locations or locations over a greater depth range are important to the user. Changing a line density or altering a lateral extent of the field of view 40 may indicate one side or sides to be more important than another side and/or the center. The center may be indicated as more important.

The indication is for a change. For example and referring to FIG. 2, the user may click and drag, enter a value, turn a knob, adjust a slider, or otherwise alter the location of the bottom 42 of the field of view 40. As another example, the user may enter a value or provide another adjustment to alter the transmit focus depth 44. Any user input or control may be used. In other embodiments, the indication is an initial setting. For example, the user inputs the focal depth 44 and field of view depth 42 to cause generation of the B-mode image.

In other embodiments, other indications are used. For example, retinal tracking is performed. A camera captures the location of the retina of the user's eyes and calculates an angle or location of viewing on the display. By determining the location or locations of most frequent viewing, an indication of location is provided. The eye movements of the user indicate the view location on the B-mode image. As another example, the user enters a region of interest 46. The user may position a box or other shape, trace on the B-mode image, or use another process to directly indicate the region of interest in the B-mode image. Rather than being a region for color flow or spectral Doppler, the region is designated for B-mode on the B-mode image.

More than one indication may be received. For example, the user alters both the focal point 44 and the depth 42 of the field of view 40. The indications are received at a same time or in sequence. The multiple indications may be used together to derive the region of interest in act 34. Alternatively, one indication overrides any other indications. For example, the last received indication is used without using any earlier received indications. As another example, a user placed region of interest marker or retinal tracking is used while any changes to the transmit frequency, depth, or focal position are ignored. The more direct indications are used instead of indications relying on assumptions.

In act 34, the region of interest is identified from the indication of the user's location of interest. For example, a processor identifies the region of interest 46 from the transmit focus, transmit frequency, and/or depth of the field of view indication.

The region of interest is a two-dimensional region. The region of interest 46 is a sub-set of the entire field of view 40 or B-mode scan region. The region of interest 46 may be less than ½, less than ⅓, or less than ¼ of the field of view 40. There are locations in the field of view 40 that are included in B-mode imaging and are not within the region of interest 46. Rather than being a two-dimensional region of interest, the region of interest may be a location (e.g., point) or line.

The location, line, or area associated with the indication is used directly or indirectly. In the case of a user entered region of interest, that region is directly identified as the region of interest. In the case of prediction from other settings or changings, the corresponding point, points, line, or lines are used indirectly. For example, the transmit focal location is set as a center of the region of interest. A region of interest of a given or predetermined size is identified as centered at the location. Alternatively, the indication maps to upper or lower parts of the field of view regardless of the specific location of the indication. For example, the region of interest 46 is positioned at or near a bottom of the field of view 40 as shown in FIG. 2 where the focal location is moved deeper, even if not moved to be within the region of interest 46.

For indicated lines, the region may cover the entire line or just parts of the line. For multiple indicated locations or lines, the region may cover all of the locations or lines or just parts.

The region of interest is of a predetermined size and/or shape. The indication is used to find the location of the predetermined region of interest. The predetermined size or shape may be based, in part, on the size and/or shape of the field of view. Alternatively, the indication may be used to determine the size or shape. For example, a change in field of view depth results in use of a smaller or larger region of interest. A greater change in the field of view depth results in use of even smaller or larger region of interest. The shape may or may not vary.

Combinations of indications may indicate the position, size, and/or shape of the region of interest 46. For example, the size is based on a change in depth 42 of the field of view 40, and the location is based on the change in focal position 44.

In one embodiment, the combination is used to determine the location. Multiple indications contribute to the calculation of the location of the region of interest 46. For example, the transmit focus change to shallower and the depth of field of view to deeper indicates a region of interest placement in a middle region. As another example, changing a lateral extent of the field of view to be larger and an indication of shallower interest provides for identifying the region of interest to be in an upper side or two regions in the upper on opposite sides. Any mapping of combinations to predicted regions of interest may be used.

The combination may be weighted. For example, one type of indication is more reliable than another. For example, a depth change may be more relevant in indicating the user's region of interest than a change in the dynamic range of the image. The locations indicated by the two indications are weighted by this reliability or priority. The weighted locations are then averaged, skewing the resulting location to be closer to the higher weighted location. The location is then used for placement of the region of interest. Alternatively, the regions appropriate for each indication are identified and then averaged or combined.

Weighting may be based on the time sequence of having received the indications. For example, more recently received indications are weighted more heavily. Any number of indications may be included, such as just the last two or three indications. Both temporal (e.g., timing) and priority weighting may be used.

In act 36, one or more B-mode imaging parameters are set as a function of the region of interest. One or more B-mode imaging parameters used for subsequent or continuing B-mode imaging are changed. During an imaging session, a patient is scanned multiple times, such as at 20-40 Hz, for minutes. The transducer is maintained against the patient during the scanning session, but may be lifted and replaced. The scanning session is for one imaging appointment of a given patient. The same ultrasound imaging system is used for scanning sessions of other patients at other times.

During the scanning session, the one or more B-mode parameters are changed. For each B-mode parameter to change, a value is altered. Any of the transmit frequency, receive frequency, number of focal locations, line density, sample density, persistence, dynamic range, overall gain, depth gain, other B-mode scan parameters, other B-mode processing parameters, or combinations thereof may be altered. By altering the setting, the resulting B-mode image may be different than if the setting were not altered.

The value is based on the region of interest. The region of interest identified is used for setting the B-mode parameter. The size, location, or size and location of the region of interest are used to determine the value. A value of the parameter is changed based on the region of interest. The region of interest may indicate a value to be used rather than an amount of change of the value. Alternatively, the region of interest is used to determine the amount of change or difference in the value.

In one embodiment, the value is selected or looked-up from a table. The location of the region of interest is used as a look-up or address to determine the value or combination of values of the B-mode parameters to use. The center, size, shape, or other characteristic of the identified region may be used. The table of predetermined values for the B-mode parameters includes various ranges or possible region positions and corresponding values. For example, a table of presets that are not just dependent on selected examination (e.g., application) and selection of B-mode, but also on the region of interest, is provided. The values are extracted using various criteria, one of which is or is derived from the region of interest.

In one embodiment, the location from the indication is used to determine the value. The value is appropriate for the corresponding region of interest with or without specifically calculating the position of the region of interest. In other embodiments, the specifically calculated region of interest is used to then map to the value or values of the parameters.

In another embodiment, the region of interest is used for automated or adaptive calculation of the value or values. The B-mode data for the region of interest and not from locations outside the region of interest are used for setting the value. For example, overall gain is set to position tissue values within a particular range of the dynamic range. The overall gain adjustment is based on the B-mode values for tissue locations. Only tissue locations in the region of interest are identified. The B-mode data for those locations are used to determine the overall gain. Any adaptive or automated process for determining the value of a B-mode parameter may be used. The adaptive or automated process uses B-mode information from within the identified region of interest for setting the value. Adaptive optimization uses the identified region. The use may be weighted rather than exclusive, such as weighting information from the region more heavily than information from outside the region. Alternatively, the automated process may use information from outside the region instead of data in the region of interest.

In act 38, another B-mode image is generated. The B-mode image is generated after the setting of the one or more B-mode parameters. Some or none of the previously used settings are used for the subsequent imaging. For example, one, two, or three B-mode parameters are changed by the user, and other B-mode parameters are altered based on the predicted region of interest. B-mode imaging with the new combination of values for the B-mode parameters is performed. Some or none of the B-mode parameters may stay the same or not change. The ultrasound imaging system scans, processes and generates an image for B-mode imaging based on the B-mode parameters. A sequence of images using the same settings may be generated.

As compared to act 30, the subsequent B-mode image is generated with at least one different value for a parameter. A value is changed or different by any amount. For example, persistence is provided to increase signal-to-noise ratio where the user is interested in viewing deeper in the field of view but not provided at all where the user is interested in shallower locations. The amount of change may or may not result in a user noticeable difference.

The settings are used for generating the entire B-mode image. The same settings are used for the entire field of view. For example, a lower or higher transmit frequency is used for scanning each transmit line in the field of view. A given value of a B-mode parameter is the same for each location in the field of view whether the location is within or outside the region of interest. The value may change or be different for different locations, such as altering depth gain. Alternatively, the value is used for the region of interest and a different value is used for locations outside the region of interest.

The B-mode image is generated without a graphic showing the region of interest. Unlike a region of interest for color flow or spectral Doppler, a graphic, coloration, or marker indicating the region of interest is not provided on the B-mode image. Instead, the B-mode image is displayed to represent the entire field of view without any indication of the region of interest. In alternative embodiments, the identified region of interest is shown on the B-mode image, such as by a box or other outline graphic of the region of interest.

FIG. 3 shows one embodiment of a system 10 for optimization in B-mode ultrasound imaging. Indications from the user of a relative importance of different parts of a B-mode image are used to optimize the B-mode imaging. The B-mode scan and processing parameters appropriate for some locations may be different than for other locations. By identifying the region of interest to the user in B-mode imaging, such as by prediction indirectly from one or more settings, the B-mode imaging may be optimized to the region of interest.

The system 10 is an ultrasound imager. In one embodiment, the ultrasound imager is a medical diagnostic ultrasound imaging system. In alternative embodiments, the ultrasound imager is a personal computer, workstation, PACS station, or other arrangement at a same location or distributed over a network for real-time or post acquisition imaging. The system 10 uses B-mode data to predict the region of interest and to inform an ultrasound system of the settings to use in B-mode imaging.

The system 10 implements the method of FIG. 1 or other methods. The system 10 includes a transmit beamformer 12, a transducer 14, a receive beamformer 16, a B-mode detector 18, a persistence filter 20, a display 22, a processor 24, a memory 26, and a user input 28. Additional, different or fewer components may be provided. For example, the receive beamformer 16 through the display 22 represents a B-mode processing path of an ultrasound imager. Other components may be provided in the path, such as a spatial filter, a scan converter, a mapping processor for setting dynamic range, or an amplifier for application of gain. While the processor 24 is shown for controlling the transmit beamformer 12, the receive beamformer 16, the B-mode detector 18 and the persistence filter 20, fewer, different, or additional components may be controlled.

The transmit beamformer 12 is an ultrasound transmitter, memory, pulser, analog circuit, digital circuit, or combinations thereof. The transmit beamformer 12 is configured to generate waveforms for a plurality of channels with different or relative amplitudes, delays, and/or phasing to focus a resulting beam at one or more depths. The waveforms are generated and applied to a transducer array with any timing or pulse repetition frequency. For example, the transmit beamformer 12 generates a sequence of pulses for different laterally and/or range regions. The pulses have a center frequency.

The transmit beamformer 12 connects with the transducer 14, such as through a transmit/receive switch. Upon transmission of acoustic waves from the transducer 14 in response to the generated waves, one or more beams are formed during a given transmit event. The beams are for B-mode imaging. For scanning tissue displacement, a sequence of transmit beams are generated to scan a one, two or three-dimensional region. Sector, Vector®, linear, or other scan formats may be used. The same region is scanned multiple times for generating a sequence of B-mode images.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. For example, the transducer 14 is a one-dimensional PZT array with about 64-256 elements.

The transducer 14 connects with the transmit beamformer 12 for converting electrical waveforms into acoustic waveforms, and connects with the receive beamformer 16 for converting acoustic echoes into electrical signals. The transducer 14 transmits the transmit beams where the waveforms have a frequency and are focused at a tissue region or location of interest in the patient. The acoustic waveforms are generated in response to applying the electrical waveforms to the transducer elements. The transducer 14 transmits acoustic energy and receives echoes. The receive signals are generated in response to ultrasound energy (echoes) impinging on the elements of the transducer 14.

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to each transmission for detection. Dynamic focusing on receive may be provided. The receive beamformer 16 outputs data representing spatial locations using the received acoustic signals. Relative delays and/or phasing and summation of signals from different elements provide beamformation. In alternative embodiments, the receive beamformer 16 is a processor for generating samples using Fourier or other transforms. The sampling by the receive beamformer 16 is for a range of depths. Timing is used to select the range of depths over which the sampling occurs.

The receive beamformer 16 may include a filter, such as a filter for isolating information at a second harmonic or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental, or other band.

The receive beamformer 16 outputs beam summed data representing spatial locations. Data for a single location, locations along a line, locations for an area, or locations for a volume are output. The data is for B-mode detection.

The processor 24 and/or a separate beamformer controller configures the beamformers 12, 16. By loading values into registers or a table used for operation, the values of acquisition parameters used by the beamformers 12, 16 for B-mode imaging are set. Any control structure or format may be used to establish the imaging sequence. The beamformers 12, 16 are caused to acquire data for B-mode imaging at a frame rate, with a transmit focus, at an imaging frequency band, over a depth, and/or with a resolution. Different values of one or more acquisition or scanning parameters may result in a different frame rate, signal-to-noise ratio, penetration, and/or resolution.

The B-mode detector 18 detects intensity from the beamformed samples. Any B-mode detection may be used. In one embodiment, the B-mode detector is a general processor, application specific integrated circuit, or field programmable gate array. Log compression may be provided by the B-mode detector so that the dynamic range of the B-mode data corresponds to the dynamic range of the display.

The persistence filter 20 is a processor, analog circuit, digital circuit, application specific integrated circuit, field programmable gate array, or other filter for temporal filtering. For example, the persistence filter 20 provides buffers or memories for storing frames of data and weights, multipliers for weighting the frames, and a summer for summing the weighted frames. Using infinite or finite impulse response, the persistence filter 20 averages two or more frames of B-mode data. Spatial filtering may be provided.

The display 20 is a CRT, LCD, monitor, plasma, projector, printer or other device for displaying an image or sequence of images. Any now known or later developed display 20 may be used. The display 20 displays two-dimensional images or three-dimensional representations. The display 20 displays one or more images representing the scan region. B-mode images are displayed.

The spatial resolution and/or image quality is based, in part, on the B-mode acquisition or B-mode processing parameters. The ultrasound imager using different acquisition and/or B-mode processing parameters may result in different spatial resolutions, temporal resolution, or image quality for the displayed image. Different transmit frequencies tradeoff between penetration depth, signal-to-noise ratio, and resolution. Different persistence trades off between signal-to-noise ratio and temporal resolution. A change in parameters of a sequence may result in images of the sequence having different spatial resolution, frame rate, temporal resolution, signal-to-noise ratio, and/or image quality. These differences may be more or less pronounced for one region relative to another region. The parameters may be optimized to provide better quality, resolution, signal-to-noise ratio or other characteristic on one location in an image relative to another location.

The ultrasound imager includes, interacts with, or is controlled by the processor 24. The processor 24 is part of a control system of the ultrasound imager or is a separate computer. The memory 26 and user input 28 are shown as associated with the processor 24, but may alternatively or additionally be part of the ultrasound imager.

The user input 28 is a track ball, mouse, joy stick, touch pad, buttons, slider, knobs, position sensor, combinations thereof or other now known or later developed input devices. The user input 28 is operable to receive a selected point, line, trace, box, other shape, value, or other information for controlling B-mode imaging by user setting of one or more B-mode parameters. For example, the user clicks and drags a focal point or part of the field of view to alter the focal depth or field of view depth. As another example, the user enters a value or selects a value from a drop down menu for one or more B-mode parameters. In yet other examples, the user rotates a knob or moves a slider to alter the values of one or more B-mode parameters.

The user input 28 is part of a user interface. The hardware and/or software of the user interface configure the user input 28 to receive inputs from the user and relate those inputs to specific B-mode parameters. The user input 28 is configured to receive an input of a region of the first B-mode image. Such input may be by prediction, such as predicting the region based on an indication of a focus of the user (e.g., receive a change in focal position, depth, or focal position and depth of a scan). This received information is used to predict or derive the region.

The processor 24 is a control processor, general processor, digital signal processor, graphics processing unit, application specific integrated circuit, field programmable gate array, network, server, group of processors, data path, combinations thereof, or other now known or later developed device or devices for associating the received user input with a region of interest and/or values of one or more B-mode parameters optimized for a focus of the user. The processor 24 is configured by software and/or hardware to perform the acts.

The processor 24 is configured to set B-mode acquisition and/or processing parameters of the beamformers 12, 16, detector 18, persistence filter 20, and/or other components of the ultrasound imager. The values of the parameters are calculated by look-up, calculated from scratch using an equation or function, calculated as an amount of alteration from a current value, and/or calculated based on feedback or adapting to B-mode data. For example, B-mode data for a sub-set of the B-mode image is used to calculate a value. The sub-set is determined based on the user focus. A region of interest to the user is found using inputs from the user.

In one embodiment, the processor 24 receives input from the user input 28. The input indicates a setting of a value of a variable used for B-mode imaging, such as indicating a change in the transmit focal position, number of foci along each scan line, change of depth of the field of view, and/or change in imaging frequency (e.g., transmit and/or receive frequency). A user set region designator or eye movement tracking may be received. The processor 24 changes the value of another variable based on the input from the user. The input from the user is used to predict part of the field of view for which the user is interested. The value of the B-mode variable is changed to provide imaging more appropriate for that part. The locations outside that part are not used for setting the value or are used to a lesser extent. For example, the value is changed based on B-mode information for the part and not any B-mode information for locations in a different part of the field of view. As another example, the value is changed using predetermined settings that are dependent on the part as indicated by the user setting.

The processor 18 causes the ultrasound imager to generate and output one or more B-mode images. The B-mode images are generated with the values for the B-mode variables set based, at least in part, on the predicted region of interest. The value of the variable, changed due to the identified region of interest of the user, is used for generating the B-mode image or images.

The processor 24 and/or the ultrasound imager operate pursuant to instructions stored in the memory 26 or another memory. The instructions configure the system for performance of the acts of FIG. 1. The instructions configure the processor 24 and/or the ultrasound imager for operation by being loaded into a controller, by causing loading of a table of values (e.g., elasticity imaging sequence), and/or by being executed. The ultrasound imager is configured to scan a field of view, process responsive data, and generate a B-mode image. The processor 24 is configured to use a user indication of a region of interest for the B-mode imaging to set or alter one or more values for one or more variables based on the indicated region of interest.

The memory 26 is a non-transitory computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The embodiments discussed above use two-dimensional imaging as an example. The indicators, imaging, changes, and/or region of interest are within a scan plane of the patient. In other embodiments, volume scanning and imaging are used. The region of interest may be indicated and used for volume scanning and/or rendering of B-mode data.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method of optimization in B-mode ultrasound imaging, the method comprising:
generating, with an ultrasound system, a first B-mode image of a scan region of a patient;
receiving, by the ultrasound system, an indication of a user's location of interest in the B-mode image;
identifying, by a processor, the region of interest from the indication of the user's location of interest, the region of interest being a sub-set of a B-mode scan region;
setting at least one B-mode imaging parameter as a function of the region of interest; and
generating, with the ultrasound system using the setting of the at least one B-mode imaging parameter, a second B-mode image of the scan region of the patient, the second B-mode image being free of a graphic showing the region of interest.

2. The method of claim 1 wherein generating the first B-mode image comprises generating with the at least one B-mode imaging parameter being a first value, and wherein generating the second B-mode image comprises generating with the at least one B-mode imaging parameters being a second value, different than the first value.

3. The method of claim 1 wherein receiving comprises receiving the indication as a change in imaging depth.

4. The method of claim 1 wherein receiving comprises receiving the indication as a change in focal position.

5. The method of claim 1 wherein receiving comprises receiving a user input region of interest shape on the first B-mode image.

6. The method of claim 1 wherein receiving comprises receiving a view location by the user of the first B-mode image from retinal tracking.

7. The method of claim 1 wherein receiving comprises receiving the indication and another indication from the user, and wherein identifying comprises assigning first and second weights to the indication and other indication, respectively, the first and second weights being based on timing of the receiving of, predetermined priority of, or both the timing and priority of the indication relative to the other indication, and determining the region of interest as function of the first and second weights.

8. The method of claim 1 wherein identifying comprises designating an area with a size and location in the B-mode scan region based on the indication.

9. The method of claim 1 wherein receiving the indication comprises receiving an indication of a point, line or area, and wherein identifying comprises assigning the region of interest as a two-dimensional region based on the location, line or area.

10. The method of claim 1 wherein setting comprises setting from a predetermined table based on the location, size, or location and size of the region of interest.

11. The method of claim 1 wherein setting comprises setting as a function of B-mode data of the first B-mode image from the region of interest and not from locations outside the region of interest.

12. The method of claim 1 wherein setting comprises setting the at least one B-mode imaging parameter as transmit frequency, receive frequency, line density, sample density, persistence, or combinations thereof.

13. The method of claim 1 wherein generating the second B-mode image comprises generating the entire second B-mode image using the setting of the at least one B-mode imaging parameter.

14. A system for optimization in B-mode ultrasound imaging, the system comprising:
an ultrasound imager comprising a B-mode detector configured to detect B-mode data, the ultrasound imager configured to generate a first B-mode image as a function of a value of a variable;
a user input configured to receive an input of a region of the first B-mode image, the region being a focus of the user;
a processor configured to change the value of the variable based on the region and cause the ultrasound imager to generate a second B-mode image as a function of the changed value of the variable.

15. The system of claim 14 wherein the ultrasound imager comprises a temporal filter, a transmit beamformer, or both, and wherein the variable is a transmit frequency of the transmit beamformer, a persistence setting of the temporal filter, or both.

16. The system of claim 14 wherein the user input is configured to receive a change in focal position, depth, or focal position and depth of a scan as the region.

17. The system of claim 14 wherein the processor is configured to change the value based on B-mode data for the region and not for locations in a different region.

18. The system of claim 14 wherein the processor is configured to change the value based on predetermined settings dependent on the region.

19. In a non-transitory computer readable storage medium having stored therein data representing instructions executable by a programmed processor for optimization in B-mode ultrasound imaging, the storage medium comprising instructions for:
receiving a user input of a change in focal position, field of view depth, an imaging frequency, or combinations thereof;
determining a region of interest that is a sub-set of a field of view for B-mode scanning, the determining of the region of interest being a function of the user input;
changing a B-mode parameter as a function of the region of interest; and
generating a B-mode image using the changed B-mode parameter, the B-mode parameter used for the entire B-mode image.

20. The non-transitory computer readable storage medium of claim 19 wherein receiving the user input comprises receiving one of the combinations, and wherein determining the region of interest comprises determining as a function of the one combination of changes with weights based on timing of the changes.
